(19) United States
(12) Patent Application Publication
(10) Pub. No.: US 2025/0000503 A1
(43) Pub. Date: Jan. 2, 2025

(54) Hook Arrangement for a Retraction System

(71) Applicant: CIVAL Medical GmbH, Tuttlingen (DE)

(72) Inventor: Ingolf DIEZ, Tuttlingen (DE)

(21) Appl. No.: 18/755,898

(22) Filed: Jun. 27, 2024

(30) Foreign Application Priority Data
Jun. 29, 2023 (DE) 10 2023 117 209.3

(51) Int. Cl. A61B 17/02; A61B 17/00

(52) U.S. Cl. CPC A61B 17/0293; A61B 2017/00477; A61B 2017/00836; A61B 2017/00862; A61B 2017/0287

(57) ABSTRACT

The invention relates to a hook arrangement for a medical retraction system and to a medical retraction system with such a hook arrangement. An exemplary embodiment of the hook arrangement has: an elongated fixation arrangement for connecting the hook arrangement to a retractor frame of a medical retraction system; and a hook element, wherein the hook element has a hook on a first side and on a second side has a connection mechanism via which the hook element is detachably connectable or connected to the fixation arrangement.

### CROSS-REFERENCE TO RELATED APPLICATIONS

The present application claims the benefit of and priority to German Patent Application No. 10 2023 117 209.3, filed Jun. 29, 2023, the entire contents of which is incorporated herein by reference.

The invention relates generally to a hook arrangement for a medical retraction system and to a medical retraction system with such a hook arrangement.

Retraction systems are known e.g. from EP 1 038 502 B1 and DE 20 2014 003 736 U. They are also termed “surgical retractor”. Retraction systems usually have what is called a “retractor frame” and hook arrangements that can be connected to the retractor frame. These hook arrangements serve to engage in a tissue of a patient by means of a preconfigured hook. Due to the engagement of the hook in the tissue and suitable connection of the hook arrangement to the retractor frame, an area to be treated, e.g. to be operated upon, is exposed and remains easily accessible for the person carrying out the treatment, e.g. the surgeon, during treatment, e.g. during an operation.

Retraction systems are usually equipped with a plurality of hook arrangements. Each of these hook arrangements has a hook that is suitable or advantageous for a certain function or application. Flexible adaptation of the hooks is not possible. Furthermore, in the event of damage to the hook arrangement, the entire hook arrangement must be replaced.

A requirement exists for an improved hook arrangement for a retraction system and an associated retraction system. In particular, a requirement exists for a more flexibly adaptable hook arrangement for a retraction system and for a more flexibly adaptable retraction system.

According to a first aspect of the invention, a hook arrangement for a medical retraction system is proposed. The hook arrangement has an elongated fixation arrangement. The hook arrangement further has a hook element. The fixation arrangement serves to connect the hook arrangement to a retractor frame of a medical retraction system. The hook element has a hook on a first side. On a second side, the hook element has a connection mechanism. The hook element can be connected detachably or is connected detachably to the fixation arrangement via the connection mechanism.

In particular, the hook element can be connected via the connection mechanism to the fixation arrangement and can be detached from the fixation arrangement without damage, in particular non-destructively. Without damage can be understood to mean that by the connection and detachment of the hook element at the fixation arrangement via the connection mechanism, no impairment or damage of any kind occurs, for example, so that all elements, in particular the hook element and/or the fixation arrangement, can continue to be used or can be used again. A simple exchange or replacement of the hook element and/or of the fixation arrangement can be achieved in this way. The fixation arrangement can also be described in short as a fixing arrangement.

The hook is provided on a first side of the hook element. The connection mechanism is provided on a second side of the hook element. The first side and the second side differ. The first and the second side can be arranged at different ends of the hook element, for example on opposed sides or ends. The detachable connection of the fixation arrangement to the hook element takes place not via the hook on the first side, but via the connection mechanism on the second side. The detachment of the connection of the fixation arrangement from the hook element takes place not via the hook on the first side, but via the connection mechanism on the second side.

The fixation arrangement can have an elastic material or be formed by an elastic material. In this way the fixation arrangement can be taken up, for example, by suitable retainers of an associated retractor frame, for example can be clamped or squeezed. The elastic material can contain a silicone or consist of a silicone. In addition or alternatively, the hook element can have a rigid material or be formed by a rigid material. The rigid material can contain a rigid plastic, for example, or consist of a rigid plastic. Good engagement in or with the tissue can be achieved due to the rigid material. Furthermore, a high hygiene standard can be attained when using a rigid plastic.

The connection mechanism can have an opening. A section of the fixation arrangement can be capable of being introduced/can be introduced into the connection mechanism via the opening. Following introduction, a section of the fixation arrangement can be taken up and/or held in the connection mechanism.

The opening can have an at least virtually circular section. In addition or alternatively, the opening can have an at least virtually slot-shaped section. For example, a section of the fixation arrangement can be introduced, e.g. inserted or pressed in, via the at least virtually circular opening. Furthermore, a section of the fixation arrangement can be introduced, e.g. pressed in or pulled in, in a pulled and/or squeezed state, for example, via the at least virtually slot-shaped opening. The fixation arrangement can be connected to the hook element via the connection mechanism in this way. In an unpulled or unsqueezed normal state, a section of the fixation arrangement introduced into the connection mechanism can be held in the connection mechanism. For example, in an unpulled or unsqueezed normal state, a section of the fixation arrangement introduced into the connection mechanism can be held in the connection mechanism by a frictional connection with the hook element in the longitudinal direction of the introduced section and the hook element and/or by a form fit with the hook element in a transverse direction, for example perpendicular to the longitudinal direction, of the introduced section and the hook element.

The slot-shaped section is designed such that in a normal state of the introduced section, the introduced section cannot slip out or fall out. In a pulled or squeezed state, a section of the fixation arrangement can be pulled out through the slot-shaped section. Furthermore, a section of the fixation arrangement can be pulled out of the at least virtually circular section. In this way the fixation arrangement can be detached from the hook element via the connection mechanism.

The fixation arrangement can have an extension mechanism. The fixation arrangement can be detachably connectable or connected via the extension mechanism to an extension arrangement. The extension arrangement can have an at least virtually identical construction and/or an at least virtually identical configuration to the fixation arrangement. For example, the fixation arrangement and the extension arrangement can match. The extension mechanism can have a projection. Furthermore, the extension mechanism can have a recess. The recess can be at least virtually circular, for example. The projection and the recess can be located on different ends or sides of the extension mechanism. For example, the fixation arrangement can be connected via the projection to the extension mechanism. The extension arrangement can be connected to the extension mechanism via the recess. A converse configuration, in which the fixation arrangement can be connected via the recess to the extension mechanism and the extension arrangement can be connected via the projection to the extension mechanism, is conceivable and configurable.

According to a second aspect, a medical retraction system is provided. The medical retraction system has a retractor frame. The medical retraction system has at least one hook arrangement according to the first aspect.

The retractor frame can have a plurality of elongated segments. The segments can be connected detachably to one another via coupling devices. The coupling devices can have locking means. The locking means can be brought into locking engagement by means of a screw.

Locking teeth and associated depressions in the coupling devices can be provided as locking means. Alternatively, surfaces roughened with sufficient graininess can also be provided in the coupling devices, which are pressed against each other by means of screws and thus come into locking engagement.

Regardless of the precise configuration, an easy-to-handle, stable coupling between the segments of the retractor frame can be enabled both with locking teeth and with rough surfaces.

The locking teeth can be arranged on a ring, for example in the manner of a rosette, such that elongated, sharp tooth cutting edges extend radially.

The same then applies to the grooves between the teeth, into which the teeth of the complementary element of the coupling device can engage.

The segments can each have a flat, plastically bendable band with humps. The humps can serve in particular to engage with the tissue to be pushed aside in the area of the operating field.

If said segments are to be capable of assembly into a closed retractor frame, a number of segments, for example, in particular four or more segments, can be used. A retractor frame can be formed in this way.

At one end, in particular centrically in relation to surrounding locking means (teeth or rough surfaces), individual segments can have an opening through which a screw can be insertable. In the coupling device of an adjacent segment, an internal thread can be provided, in particular centrically in relation to the locking means (teeth or rough surfaces), in such a way that the screw can be screwed with its thread into the internal thread.

The at least one hook arrangement can have a plurality of hook arrangements according to the first aspect or be formed as a plurality of hook arrangements according to the first aspect. Expressed another way, a plurality of hook arrangements according to the first aspect can be provided in the retraction system. A hook element of a first of the plurality of hook arrangements can have a hook with a first hook shape. A hook element of a second of the plurality of hook arrangements can have a hook with a second hook shape differing from the first hook shape. A hook element of a third of the plurality of hook arrangements can have a hook with a third hook shape differing from the first and/or second hook shape or can have a hook with a third hook shape corresponding to the first and/or second hook shape. This list of hook elements with identical or differing hook shapes can be continued accordingly, depending on the purpose of use. Overall a plurality of hook shapes of the respective hooks of the hook elements can be provided that at least partially differ from one another and/or at least partially correspond.

A plurality of hook elements can be provided. The fixation arrangement can be detachably connectable or connected optionally to one of the plurality of hook elements via the connection mechanism described. Expressed another way, the at least one hook arrangement can be provided detachably optionally with one of the plurality of hook elements via the connection mechanism described. The hook arrangements can be provided optionally with one of the plurality of hook elements in this way. In the event of damage, an exchange of the hook element and/or the fixation arrangement is possible. An exchange of the hook element is likewise possible if another application should be desired.

The at least one hook arrangement can be connectable detachably to the retractor frame via the respective fixation arrangement. The retractor frame can thereby be provided with one or more hook arrangements. In this way the retraction system can be equipped for the respective specific application. If another hook element should be desired, the entire hook arrangement does not need to be exchanged, but rather just a new hook element can be arranged on an already present fixation arrangement. This can increase the flexibility and/or reduce the consumption of hook arrangements and/or increase the speed in the event of an exchange. If a fixation arrangement is damaged during treatment, for example during an operation, the entire hook arrangement does not have to be exchanged—which is at least virtually impossible in many cases, as the associated hook is often anchored in the tissue—but rather the damaged fixation arrangement can be detached from the hook element and replaced by a new, intact fixation arrangement.

Even if some of the aspects described above were described in relation to the hook arrangement according to the first aspect, these aspects can also be realised in a corresponding manner in the retraction system according to the second aspect.

The present invention is to be explained further on the basis of figures. These figures show schematically:

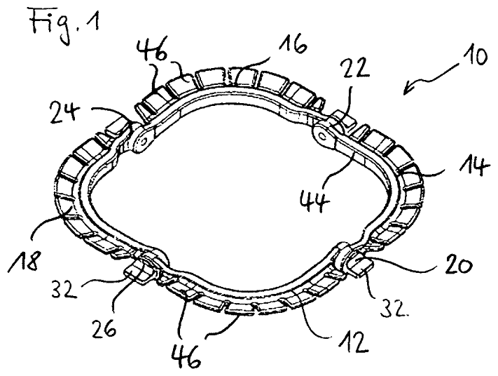

FIG. 1 an example of a retractor frame in a perspective view;

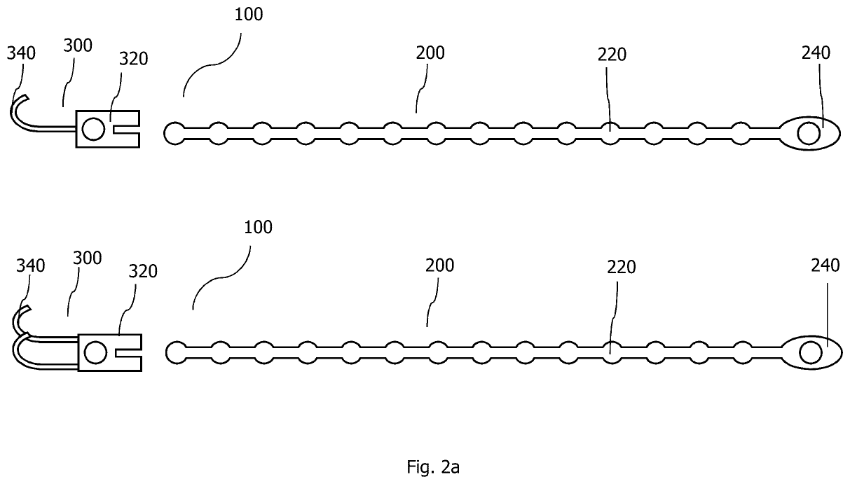

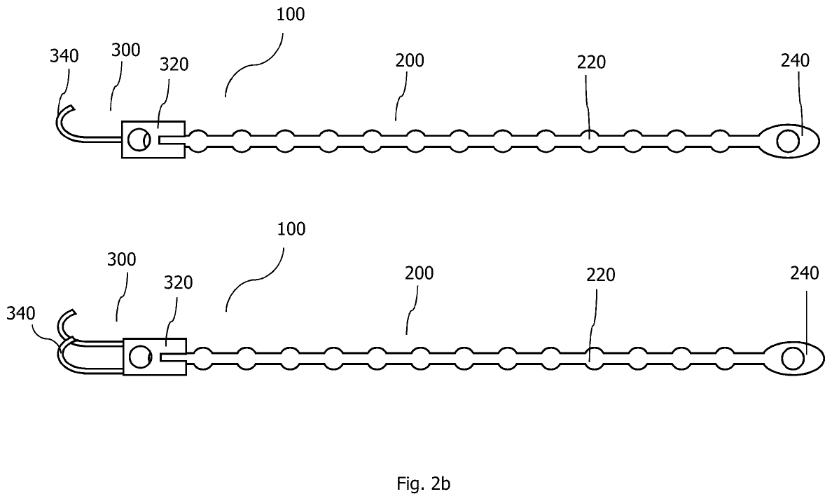

FIG. 2A two exemplary embodiments of a hook arrangement in an unconnected state;

FIG. 2B two exemplary embodiments of the hook arrangement from FIG. 2a in a connected state;

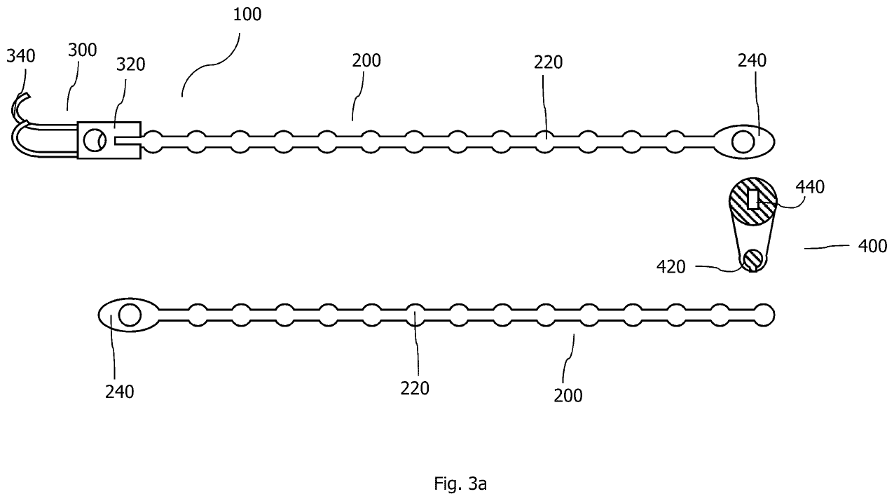

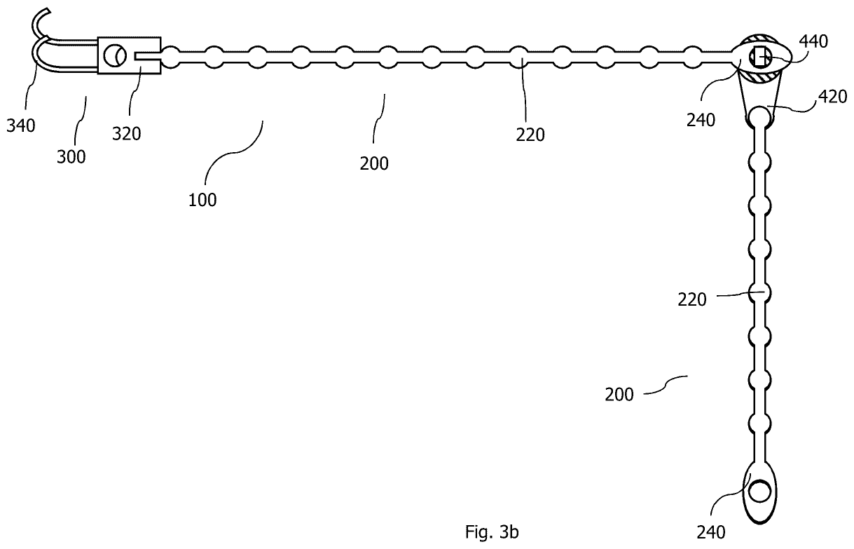

FIG. 3A a variant of one of the exemplary embodiments of a hook arrangement from FIG. 2a/2b in a connected state with an unconnected extension;

FIG. 3B a variant of the hook arrangement from FIG. 3a in a connected state with a connected extension; and

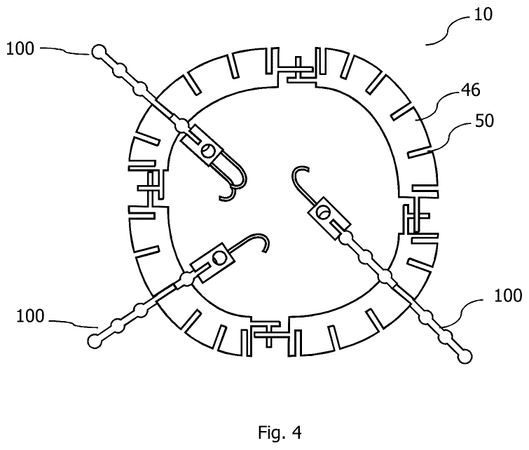

FIG. 4 an exemplary embodiment of a retraction system with a retractor frame and a number of hook arrangements according to the exemplary embodiments from FIGS. 2a to 3b.

In the following, without being restricted hereto, specific details are explained in order to provide a complete understanding of the present invention. It is clear to an expert, however, that the present invention can be used in other exemplary embodiments that may differ from the details explained below. The figures serve further purely for the purpose of clarifying exemplary embodiments. They are not true to scale and are only intended to reflect the general concept of the invention by way of example. For example, features that are contained in the figures are by no means to be considered a necessary constituent.

In FIG. 1, a possible/applicable retractor frame 10 is shown by way of example. The retractor frame 10 according to FIG. 1 has four segments 12, 14, 16, 18, which are closed by means of coupling devices 20, 22, 24, 26 to form a ring. For example, the segment 12 has a ring at one end and the segment 14 has a ring at one end. A wing screw 32 with an external thread is pushed through a hole in the ring of the segment 14 and engages by screwing in an internal thread in the opposing ring of the segment 12. The ring of the segment 12 is provided on its surface facing the ring of the segment 14 with locking teeth 34, which run radially with their sharp tip lines in relation to the axis running through the hole and the internal thread (the same then applies to the depressions (grooves) between the teeth).

According to a supplementation, not shown, of the retractor frame, a simple spring system, e.g. a leaf spring or similar, can be provided between the rings of the segments 12, 14 on one of the rings, which system automatically causes the rings of the adjacent segments 12, 14 to disengage from one another when the screw 32 is loosened.

The individual segments 12, 14, 16, 18 each have an elongated band 44, which consists of a suitable plastic and is deformable such that following deformation it assumes the desired configuration sufficiently stably to press tissue out of the operating field during operations. For this purpose, humps 46 are arranged directed outwardly along the bands 44 that can be bent in relation to the bands in order to be brought into optimal engagement with the tissue.

Even if a specific retractor frame was described in detail in relation to FIG. 1 by way of example, the hook arrangements described below can be used not only with this specific retractor frame 10, but with a plurality of different retractor frames if these retractor frames permit the creation of a connection between the hook arrangements used and the retractor frame used.

In FIG. 2a, two hook arrangements 100 for a medical retraction system are shown. The two hook arrangements 100 each have an elongated fixation arrangement 200 for connecting the hook arrangement 100 to a retractor frame 10 of a medical retraction system. In exemplary use of the hook arrangements 100 with the retractor frame 10 shown in FIG. 1, the respective fixation arrangement 200 of the hook arrangements 100 can be arranged and/or fixed and/or clamped in gaps or spacings or slots between the aforesaid humps 46 of the retractor frame 10.

Furthermore, the two hook arrangements 100 each have a hook element 300. Each hook element 300 has a hook 340 on a first side and a connection mechanism 320 on a second side. The hook element 300 is detachably connectable via the connection mechanism 320 to the fixation arrangement 200. In FIG. 2a, the respective hook element 300 and the respective fixation arrangement 200 are not connected to one another. The two hook arrangements 100 shown in FIG. 2a substantially correspond to one another. Put more precisely, the two hook arrangements 100 correspond exactly except for the hook element 300. Put even more precisely, the two hook arrangements 100 correspond exactly except for the hook 340 (in particular the hook shape) of the hook element 300. The hook 340 of the upper hook element 300 is formed by a single hook. The hook 340 of the lower hook element 300 is formed by a double hook. On account of the conformities, reference is made in the following in part only to a single hook arrangement 100 in the case of the corresponding elements; the description applies accordingly, however, to the corresponding elements of the other hook arrangement 100.

The fixation arrangement 200 in the example shown is formed from a silicone. Both hook elements 300 in the example shown are formed from a rigid plastic.

The connection mechanism 320 has an opening, via which a section of the fixation arrangement 200 can be introduced into the connection mechanism 320. In the example shown, the opening has an at least virtually circular section (only described in short as a circular section below) and an at least virtually slot-shaped section (only described in short as a slot-shaped section below) connecting thereto (to the at least virtually circular section). The fixation arrangement 200 has a plurality of at least virtually spherical or ball-shaped bulges 220. The bulges 220 are convex or raised compared with the remaining sections of the fixation arrangement. The foremost bulge 220 (i.e., the left-hand bulge 220 in FIG. 2a), can be inserted or pressed from above into the circular section 320. Although the diameter of the bulges 220 is slightly larger than the diameter of the circular section of the opening, due to the exemplary formation of the fixation arrangement 200 from silicone, the bulge can be (easily) compressed or squeezed through the circular section and thereby pressed or squeezed through the circular section. In this way, the foremost bulge 220 can be pressed into the interior of the hook element 300, stated more precisely into the interior of the connection mechanism 320 and taken up there.

The straight, tube- or cylinder-shaped section of the fixation arrangement 200 connected to the foremost bulge 220 can be pressed or pulled through the slot-shaped section. In this way, a section of the fixation arrangement 200 containing the foremost bulge 220 and the straight, tube- or cylinder-shaped section connected thereto is taken up in the connection mechanism 320. If the tube- or cylinder-shaped section of the fixation arrangement 200 is pulled through the slot-shaped section of the opening, the foremost bulge 220 is pulled furthermore contrary to the direction pointing in the direction of the hook 340. The fixation arrangement 200 and the hook element 300 are thereby connected to one another, as is shown in FIG. 2b. Between the hook element 300, stated more precisely inner faces of the opening of the connection mechanism 320, and the foremost bulge 220 and the tube- or cylinder-shaped section of the fixation arrangement 200 connected thereto, a radially acting form fit and a friction connection acting in a longitudinal direction are created.

Since the length of the slot-shaped section is coordinated to the length of the (tube- or cylinder-shaped) section of the fixation arrangement 200 connected to the foremost bulge 220, these at least virtually correspond, the second-foremost bulge 220 rests—following the connection of fixation arrangement 200 and hook element 300—directly on one side of the hook element 300 and in the direction of the hook element 300 furthermore creates with the hook element 300 a form fit in a longitudinal direction. Due to the connection described, a stable yet detachable connection is produced between the hook element 300 and the fixation arrangement 200.

The connection can be detached again in that the fixation arrangement 200 is pulled on its unconnected side upwards and/or in the direction of the hook 340 and thereby the straight (tube- or cylinder-shaped) section is at least partially detached from the slot-shaped section. The foremost bulge 220 remains as a rule initially still taken up in the hook element 300. By pulling the fixation arrangement 200 further in the direction of the hook element 300, the foremost bulge 220 moves in the direction of the hook 340, enters the region of the circular section and can be pulled through this out of the connection mechanism 320 and the hook element 300. In this way the connection between the fixation arrangement 200 and the hook element 300 can be detached without damage, in particular non-destructively.

In FIG. 3a, a variant of the example from FIGS. 2a and 2b is shown. In this variant, the fixation arrangement 200 has an extension mechanism 400. Put more precisely, the fixation arrangement 200 in the example shown is connectable detachably to an extension mechanism 400. This means that in the example of FIG. 3a, the fixation arrangement 200 and the extension mechanism 400 are formed as separate elements, i.e. in two parts. Alternatively, the fixation arrangement 200 and the extension mechanism 400 can also be formed in one piece. The fixation arrangement 200 is detachably connectable or connected via the extension mechanism 400 to an extension/extension arrangement. The extension mechanism 400 has on one side a projection 440 and on the other side an at least virtually circular recess 420 (circular recess in short below). The projection 440 can be pressed through a corresponding opening 240 in the fixation arrangement 200 in order to connect the fixation arrangement 200 and the extension mechanism 400 detachably to one another. The extension/extension arrangement in the example shown is formed corresponding to the fixation arrangement 200, i.e. a second fixation arrangement 200 is present, and is provided and described accordingly with the associated reference characters. The foremost bulge (the right-hand bulge 220 in FIG. 3a) of the second fixation arrangement 200 (=extension arrangement) can be pressed into the circular recess 420 and held therein. In this way, the second fixation arrangement 200 (=extension arrangement) can be connected detachably to the extension mechanism 400. An extended hook arrangement 100 is created thereby.

FIG. 3b shows the connected extension state, i.e. in FIG. 3b the fixation arrangement 200 is connected—by connecting the projection 440 to the opening 240—to the extension mechanism 400 and the second fixation arrangement 200 (=extension arrangement) is connected—by take-up of the right-hand bulge 220 in the circular recess 420—to the extension mechanism 400. The fixation arrangement 200 is therefore connected via the extension mechanism 400 to the second fixation arrangement 200 (=extension arrangement). For detachment, the opening 240 can be pulled off the projection 440 and the right-hand bulge 220 can be pulled out of the recess 420.

FIG. 4 shows a medical retraction system. The retraction system has a retractor frame 10, and in the example shown three hook arrangements 100. The basic construction of the retractor frame from FIG. 4 corresponds substantially to the basic construction of the retractor frame 10 from FIG. 1, even if details may differ from one another. Even if only three hook arrangements 100 are shown by way of example, any number of hook arrangements 100 can be provided, for example only one hook arrangement 100 or two or more than three hook arrangements 100. The three hook arrangements can be connected detachably via the respective fixation arrangement 200 to the retractor frame 10 and are each connected in the example shown in FIG. 4. The connection takes place in that the fixation arrangement 200, more precisely one of the tube- or cylinder-shaped sections of the fixation arrangement 200, is clamped in a gap 50 of the plurality of gaps 50, which are each located between two humps 46.

The fixation arrangement is held and/or fixed by a friction connection acting in the longitudinal direction of the fixation arrangement 200 with the adjoining sides of the humps 46 and a form fit with the humps 46 acting transversely to the longitudinal direction of the fixation arrangement 200. Greater shifting of the fixation arrangement 200 is prevented by the bulges 220.

In the example shown in FIG. 4, one hook element 300 of the three hook arrangements 100 is formed as a single hook as hook shape, one hook element 300 of the three hook arrangements 100 is formed as a single hook as hook shape and one hook element 300 of the three hook arrangements 100 is formed as a double hook as hook shape. This is to be understood as purely exemplary. The hook elements 300 can be optionally interchanged and connected to another fixation arrangement 200.

Furthermore, the hook elements 300 can easily be replaced by a hook element of another hook shape as described above, e.g. if another purpose of use of the hook element 300 is to be achieved. In the event of damage, a hook element 300 can also be replaced by another, undamaged hook element 300 of the same or different hook shape, as described above.

Furthermore, in the event of damage, a fixation arrangement 200 can be replaced by another undamaged fixation arrangement 200, as described above. The hook element 300 can then remain in place and be connected to the undamaged fixation arrangement 200.

The details described with regard to the figures provide an improved, in particular flexibly adaptable, hook arrangement 100 and an improved, in particular flexibly adaptable, retraction system with such a hook arrangement 100.

1. A hook arrangement for a medical retraction system, having:
an elongated fixation arrangement for connecting the hook arrangement to a retractor frame of a medical retraction system; and
a hook element, wherein the hook element has on a first side a hook and on a second side has a connection mechanism, via which the hook element is detachably connectable or connected to the fixation arrangement.

2. The hook arrangement according to claim 1, wherein the fixation arrangement has an elastic material or is formed by an elastic material, for example a silicone, and/or the hook element has a rigid material or is formed by a rigid material, for example a rigid plastic.

3. The hook arrangement according to claim 1, wherein the connection mechanism has an opening, via which a section of the fixation arrangement can be introduced into the connection mechanism.

4. The hook arrangement according to claim 3, wherein the opening has an at least virtually circular section.

5. The hook arrangement according to claim 3, wherein the opening has an at least virtually slot-shaped section.

6. The hook arrangement according to claim 1, wherein the fixation arrangement has an extension mechanism, via which the fixation arrangement is detachably connectable or connected to an extension arrangement.

7. A medical retraction system having:
a retractor frame; and
at least one hook arrangement according to claim 1.

8. The medical retraction system according to claim 7, wherein the at least one hook arrangement has a plurality of hook arrangements, wherein a hook element of a first of the plurality of hook arrangements has a hook with a first hook shape and a hook element of a second of the plurality of hook arrangements has a hook with a second hook shape differing from the first hook shape.

9. The medical retraction system according to claim 7, wherein a plurality of hook elements is provided and a fixation arrangement of the at least one hook arrangement is detachably connectable via the connection mechanism optionally to one of the plurality of hook elements.

10. The medical retraction system according to claim 7, wherein the at least one hook arrangement is connectable via the respective fixation arrangement detachably to the retractor frame.
